(19) United States
(12) Patent Application Publication
(10) Pub. No.: US 2012/0303067 A1
(43) Pub. Date: Nov. 29, 2012

(54) Elbow Compression Instrument

(76) Inventors: PETER CHARLES VAN CITTERS, WEST CHESTER, PA (US); KENNETH ISAMU KOBAYASHI, WEST CHESTER, PA (US)

(21) Appl. No.: 13/114,667

(22) Filed: May 24, 2011

(51) Int. Cl. A61B 17/66; A61B 17/80; A61B 17/84; A61B 17/17; A61B 17/16

(52) U.S. Cl. 606/281; 606/105; 606/96; 606/80; 606/300

(57) ABSTRACT

A device for compressing fractured portions of a bone includes (a) first and second jaws movable relative to one another to compress fractured portions of a bone therebetween, the first and second jaws being connected to one another such that bone gripping portions thereof remain parallel to one another throughout a permitted range of motion of the first and second jaws and (b) a first guide sleeve attached to the distal end of the first jaw and a second guide sleeve attached to the distal end of the second jaw. The first and second guide sleeves have central axes coaxial with one another.

### BACKGROUND

Fractured fragments of a bone may be brought together using, for example, surgical forceps. Currently, surgical forceps include a pair of jaws that pivot around a central hinged joint such that it is often difficult to achieve proper compression of the fragments. In addition, a combination of multiple devices must be used to compress the fragments, hold the compressed fragments in a desired position relative to one another and to guide drills, bone fixation elements and other tools through one or more holes of a bone plate positioned along the fractured bone to permanently fix the fragments in position.

### SUMMARY OF THE INVENTION

The present invention relates to a device for compressing fractured portions of a bone, comprising first and second jaws movable relative to one another to compress fractured portions of a bone therebetween, the first and second jaws being connected to one another such that bone gripping portions thereof remain parallel to one another throughout a permitted range of motion of the first and second jaws and a first guide sleeve attached to the distal end of the first jaw and a second guide sleeve attached to the distal end of the second jaw, the first and second guide sleeves having central axes coaxial with one another.

### BRIEF DESCRIPTION OF THE DRAWINGS

FIG. 1 shows a top plan view of a device according to an exemplary embodiment of the present invention;

FIG. 2 shows a perspective view of a first handle of the device of FIG. 1;

FIG. 3 shows a perspective view of a second handle of the device of FIG. 1;

FIG. 4 shows a perspective view of a jaw of the device of FIG. 1;

FIG. 5 shows a side view of the jaw of FIG. 4;

FIG. 6 shows a side view of a variable angle tip according to an exemplary embodiment of the present invention;

FIG. 7 shows a perspective view of a drill sleeve of the variable angle tip of FIG. 6;

FIG. 8 shows a cross-sectional view of the drill sleeve of FIG. 7;

FIG. 9 shows a perspective view of a cap of the variable angle tip of FIG. 6;

FIG. 10 shows a cross-sectional view of the cap of FIG. 9;

FIG. 11 shows a side view of a body of the variable angle tip of FIG. 6;

FIG. 12 shows a cross sectional view of the body of FIG. 11; and

FIG. 13 shows a side view of a device according to another exemplary embodiment of the present invention.

### DETAILED DESCRIPTION

The present invention may be further understood with reference to the following description and the appended drawings, wherein like elements are referred to with the same reference numerals. The present invention relates to a device for treating fractures and, in particular, to a device for compressing portions of a fractured bone. Exemplary embodiments of the present invention describe a device including a pair of parallel jaws configured to compress fractured portions of a bone such as, for example, an end of the humerus forming the elbow joint. In addition, the parallel jaws of the device maintain a desired compression of the fractured portions of bone and provide a guide for drilling a hole into a bone at an angle desired for insertion of a bone fixation element. Although the exemplary embodiments specifically describe a fractured end of a humerus, it will be understood by those of skill in the art that the device may be used to compress fractured portions of any of a variety of bones. It should be noted that the terms “proximal” and “distal” as used herein with reference to the device, are intended to refer to a direction toward (proximal) and away from (distal) a user of the device.

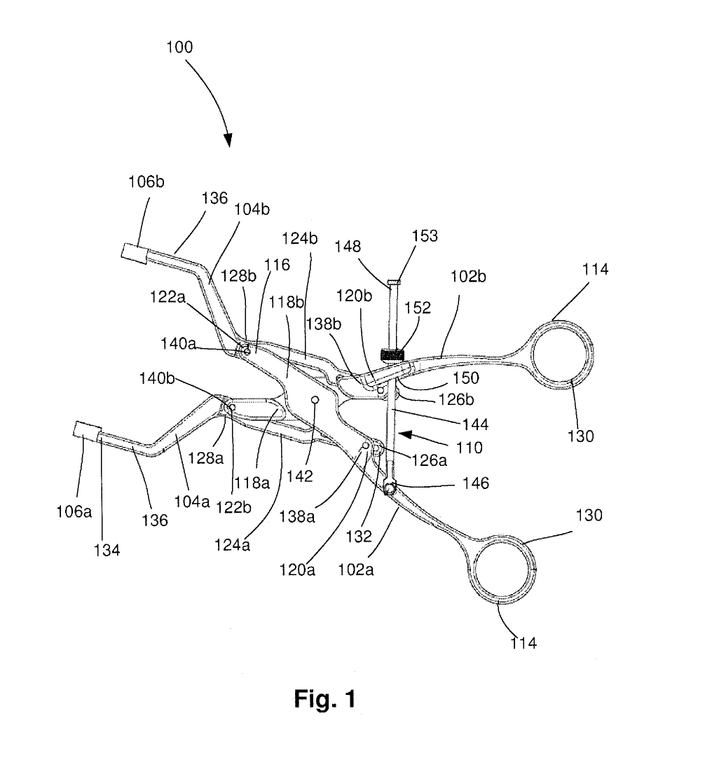

As shown in FIGS. 1-5, a device 100 according to an exemplary embodiment of the present invention comprises a pair of handles 102a, 102b and a pair of jaws 104a, 104b connected to one another such that the jaws 104a, 104b maintain a parallel configuration as the jaws 104a, 104b are moved toward and away from one another to press fractured portions of a bone (e.g., an end of the humerus forming the elbow joint) toward one another. The jaws 104a, 104b include guide sleeves 106a, 106b, respectively, at distal ends 116 thereof. Each of the guide sleeves 106a, 106b includes an opening sized and shaped to receive a guide wire, drill or other tool therethrough and, due to the configuration which maintains the ends of the jaws 104a, 104b parallel to one another, the openings in the guide sleeves 106a, 106b remain aligned with one another at all times. The device 100 also includes a locking mechanism 110 operable once the fractured portions of bone have been placed in a desired spatial relation to one another, to hold the jaws 104a, 104b at a desired distance from one another to maintain the fractured portions in the desired compressed configuration.

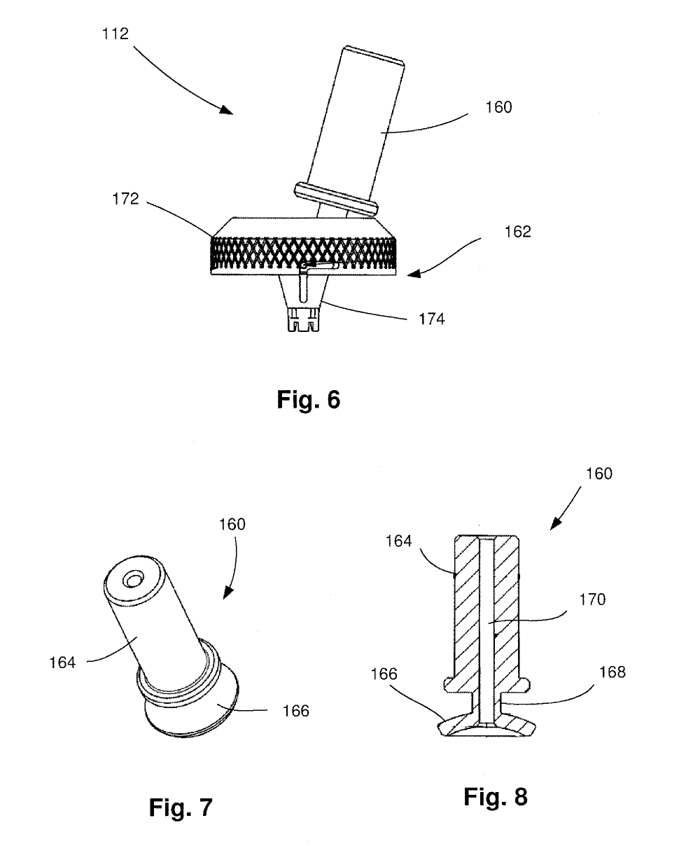

The device 100 may further include one or more variable angle tips 112, as shown in FIGS. 6-12, coupled to the guide sleeves 106a, 106b for use in conjunction with a bone plate including variable angle holes (i.e., holes which permit a bone fixation element such as a bone screw to be locked therein at an angle relative to an axis of the hole in the bone plate). The variable angle tips 112 restrict angulation of the guide sleeves 106a, 106b relative to the bone plate hole axis to a permitted range of angulation within which a bone fastening member may be locked within the variable angle hole of the bone plate. In particular, the variable angle tip 112 includes a drill sleeve 160 couplable to the guide sleeve 106a, 106b substantially coaxially with a central axis of the guide sleeve 106a, 106b and a body 162 pivotable relative thereto. The body 162 includes a tip portion 174 sized and shaped for insertion into a variable angle hole and a tapered channel 154 for guiding a guide wire and/or drill therethrough at an angle within the permitted range of angulation of the variable angle hole of the bone plate.

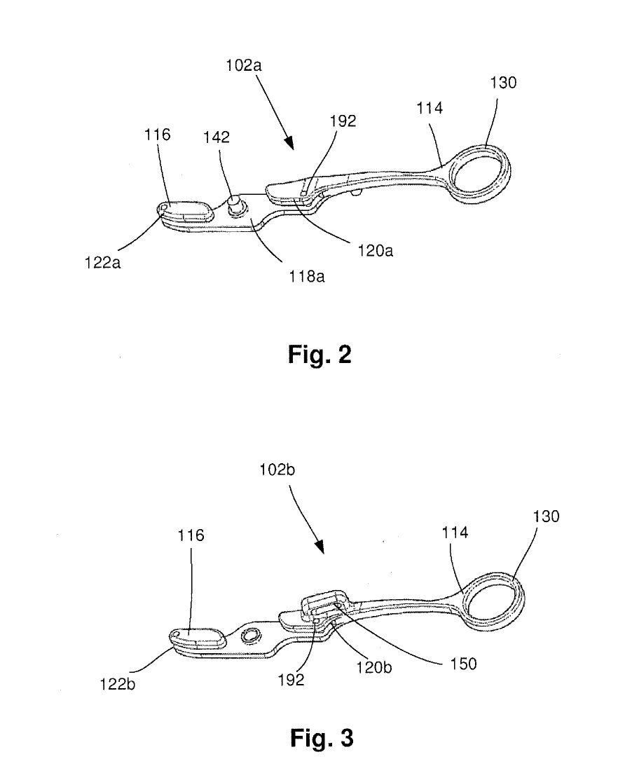

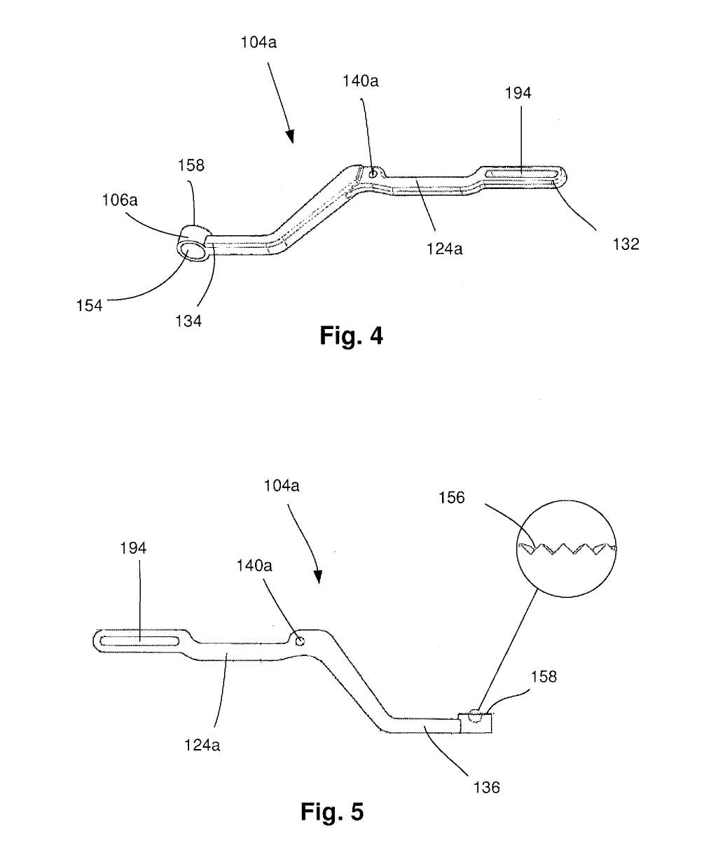

Each of the handles 102a, 102b, as shown in FIGS. 2 and 3, extends from a proximal end 114 to a distal end 116 and includes a connecting portion 118 at its distal end 116. The proximal ends 114 of the handle 102a, 102b, include gripping elements such as, for example, finger loops 130. Each of the jaws 104a, 104b, as shown in FIGS. 4 and 5, extends from a proximal end 132 to a distal end 134 and includes a corresponding connecting portion 124 at the proximal end 132, which connects the connecting portion 118 to a bone gripping portion 136 at the distal end 134. The bone gripping portions 136 are spaced farther apart from one another than the corresponding connecting portions 124 such that the gripping portions 136 may accommodate the fractured portions of the bone therebetween. As described above, the gripping portions 136 remain parallel relative to one another and maintain a parallel configuration as the jaws 104a, 104b are moved toward and away from one another. It should be noted that though FIGS. 4 and 5 show the jaw 104a, the jaw 104b includes the same elements.

The jaws 104a, 104b are placed over fractured portions of a bone and moved toward one another to compress the bone therebetween to draw the fractured portions of bone into a desired spatial relation to one another. Once the portions of the bone have been compressed to the desired relation, this desired compression is maintained via the locking mechanism 110 which holds the jaws 104a, 104b in the desired position relative to one another. The locking mechanism 110 includes a longitudinal rod 144 and a nut 152 longitudinally movable therealong. The rod 144 extends longitudinally from a first end 146 to a second end 148. The first end 146 is pivotably attached to the first handle 102a at a position proximal of the connecting portion 118a. The second end 148 includes a stop 153 extending radially outward therefrom to prevent the nut 152 from becoming disengaged from the rod 144. The second handle 102b includes an elongated opening 150 extending along a length thereof, sized and shaped to slidably accommodate the rod 144. The elongated opening 150 permits the rod 144 to be longitudinally and laterally moved therein as the handles 102a, 102b are moved toward and away from one another.

The nut 152 is movable between the stop 153 at the second end 148 of the rod 144 and the elongated opening 150 in the second handle 102b from an unlocked position to a locked position. The nut 152 may be longitudinally moved along the rod 144 via, for example, spinning engagement of an external threading along a length of the rod 144 and a corresponding internal threading along an interior of the nut 152. In the unlocked position, the nut 152 is positioned adjacent the stop 153 such that the handles 102a, 102b are movable relative to one another. As the handles 102a, 102b are moved relative to one another, the rod 144 slides through the elongate opening 150. Once sufficient movement of the handles 102a, 102b has been achieved so that the jaws 104a, 104b are in the desired position relative to one another, the device 100 may be locked by moving the nut 152 toward the first end 146 of the rod 144 until the nut abuts the second handle 102b. This prevents the handles 102a, 102b and, consequently, the jaws 104a, 104b from being moved apart from one another. Thus, the bone remains compressed between the jaws 104a, 104b.

The connecting portions 118 of the handles 102a, 102b cross over one another to attach to the jaws 104a, 104b. First and second connecting portions 118a, 118b are pivotably connected to one another via, for example, a pin 142 at the point at which the connecting portions 118a, 118b cross over one another. A proximal end 120a of the first connecting portion 118a of the first handle 102a is connected to a proximal end 126a of a first corresponding connecting portion 124a of a first jaw 104a via a sliding joint 138a while a distal end 122a of the first connecting portion 118a is pivotably coupled to a distal end 128b of a second corresponding connecting portion 124b of a second jaw 104b via a pin 140a. Similarly, a proximal end 120b of the second connecting portion 118b of a second handle 102b may be coupled to a proximal end 126b of the second corresponding connecting portion 124b via a slidable joint 138b while a distal end 122b of the second connecting portion 118b is coupled to a distal end 128a of the first corresponding connecting portion 124a via a pin 140b. The slidable joints 138a, 138b may include, for example, a pin 192 extending from the proximal ends 120a, 120b of the connecting portions 118a, 118b slidably received within an elongated opening 194 extending longitudinally through the proximal ends 126a, 126b of the corresponding connecting portions 124a, 124b. The connection between the connecting portions 118 and the corresponding connecting portions 124 of the handles 102a, 102b and the jaws 104a, 104b via the slidable and pivotable joints 138a, 138b, 140a, 140b, 142 permit movement of the handles 102a, 102b toward and away from one another so that the jaws 104a, 104b toward and away from one another while maintaining the parallel configuration of the jaws 104a, 104b.

The guide sleeves 106a, 106b are attached to the distal ends 116 of the jaws 104a, 104b such that channels 154 extending therethrough have central axes substantially perpendicular to the bone gripping portions 136 of the jaws 104a, 104b and substantially coaxial with one another. Since the jaws 104a, 104b remain parallel through the entire range of motion, the central axes of the guide sleeves 106a, 106b also remain coaxial through the range of motion. The guide sleeves 106a, 106b may also include teeth 156, or other bone engaging structures, along surfaces 158 thereof, which face a bone to be gripped therebetween. Thus, when the device 100 is used to compress fractured portions of bone, the teeth 156 grip the bone, preventing the device 100 from being inadvertently moved out of the desired position. In an alternate embodiment, the device 100 further comprises a spike couplable to one of the guide sleeves 106a, 106b such that a tip of the spike engages the bone and/or an opening of a bone plate positioned over the bone. The spike may, for example, be coupled to the first guide sleeve 106a to engage the bone and/or an opening of a bone plate positioned over the bone while the bone is being drilled via the second guide sleeve 106b.

The variable angle tip 112, as shown in FIGS. 6-12, may be coupled to the guide sleeves 106a, 106b to restrict angulation of the guide sleeves 106a, 106b relative to an axis of a variable angle hole in a bone plate positioned over the bone to a permitted range of angulation within which a bone fastening member may be locked within the variable angle hole. The variable angle tip 112 includes a drill sleeve 160 and a body 162 pivotable with respect to one another. As shown in FIGS. 7-8, the drill sleeve 160 includes a first portion 164 sized and shaped to be inserted through the guide sleeve 106a, 106b and a rounded second portion 166 for pivotably coupling the drill sleeve 160 to the body 162. The first portion 164 may engage the guide sleeve 106a, 106b via, for example, a friction fit. The first and second portions 164, 166 may be connected to one another via a smaller diameter neck portion 168. The drill sleeve 160 also includes a channel 170 extending longitudinally therethrough. The channel 170 is sized and shaped to receive a guide wire, drill tip or other bone engaging structure therethrough as would be understood by those skilled in the art.

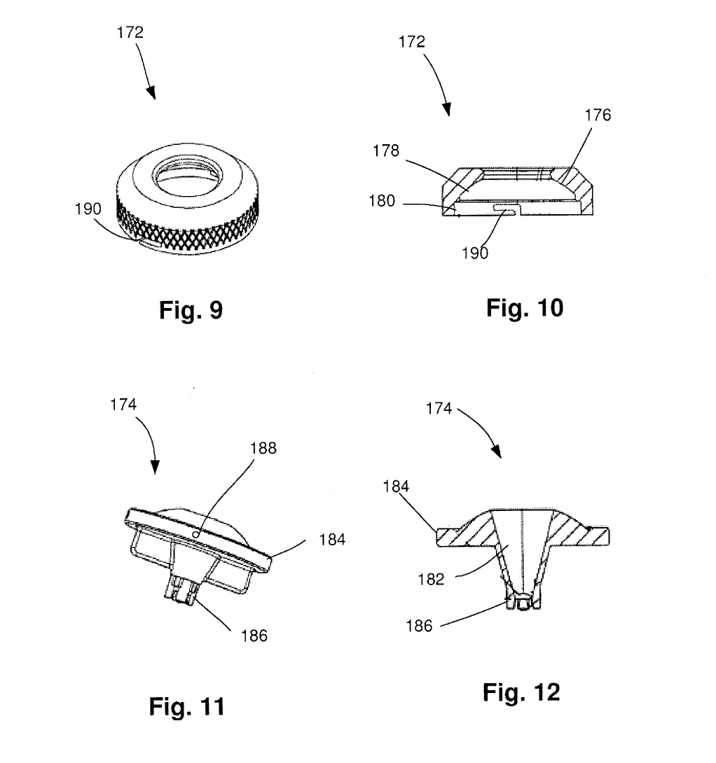

The body 162 may further include a cap 172, as shown in FIGS. 9-10, and a tip portion 174, as shown in FIGS. 11-12, coupled to one another. An inner surface 176 of the cap 172 includes a first portion 178 which mates with the rounded second portion 166 to permit the body 162 and the drill sleeve 160 to pivot with respect to one another and a second portion 180 which is sized and shaped to receive the tip portion 174. The tip portion 174 includes a channel 182, tapering from a first end 184 to a second end 186 of the tip portion 174 to permit a guide wire and/or drill tip to be inserted therethrough within a range of permitted angles relative to a central axis thereof. The first end 184 of the tip portion 174 is sized and shaped to be received within the cap 172 while the second end 186 is sized and shaped to be inserted through a variable angle hole of a bone plate. Thus, a guide wire and/or drill tip may be inserted coaxially through the drill sleeve 160 and the guide sleeve 106a, 106b at an angle with respect to the central axis of the tip portion 174 such that the guide wire and/or drill tip pass through the variable angle hole of the bone plate at any selected angle within the permitted range of angulation. The cap 172 and the tip portion 174 may be releasably coupled to one another via, for example, a pin 188 extending from the first end 184 of the tip portion 174 received within the cap 172 and a notch 190 formed along the cap 172 for receiving the pin 188. The cap 172 and the tip portion 174 may be releasably coupled to facilitate disassembly of the variable angle tip 112 for easy cleaning of the parts as would be understood by those skilled in the art.

In use, fractured portions of a bone are reduced by placing the jaws 104a, 104b over the fractured portions and moving the jaws 104a, 104b toward one another via the handles 102a, 102b to compress the fractured portions of bone therebetween until a desired spatial relationship between the fractured portions has been achieved. If it is desired to maintain the compression of the fractured portions, the jaws 104a, 104 may be locked over the bone by moving the nut 152 of the locking mechanism 110 from the unlocked position along the rod 144 to the locked position. The bone may then be temporarily fixed using, for example, K-wires inserted through the bone via, for example, the guide sleeves 106a, 106b. A bone plate may be positioned over the fractured portions of the bone and engaged by one of the jaws 104a, 104b, while the other jaw contacts an opposite surface of one of the fractured portions of bone to reduce the fracture as desired. One or more holes may then be drilled into the portions of bone through holes in the bone plate guided by the guide sleeves 106a, 106b so that the plate may be fixed to the fractured portions bone by inserting one or more bone fixation elements through the plate holes as would be understood by those skilled in the art. Once the fractured portions of the bone have been fixed in the desired position, the device 100 may be removed from the bone. Although the exemplary surgical technique only describes one bone plate, it will be understood by those of skill in the art that the bone may be fixed using more than one bone plate.

If the bone plate includes variable angle holes, a user may couple a variable angle tip 112 to the one of the first and second guide sleeves 106a, 106b which is to engage the bone plate. The above process is repeated with the hole being drilled through the variable angle hole and into the portions of bone at an angle relative to the axis of the variable angle hole which is within the permitted range of angulation for the hole. As would be understood by those skilled in the art, where two bone plates are being fixed the opposite sides of the fractured portions of bone, it may be desirable to couple a variable angle tip 112 to each of the guide sleeves 106a, 106b. As described above, the variable angle tip 112 is coupled to the guide sleeve 106a, 106b by inserting the drill sleeve 160 into the channel 154 such that, in the operative position, the body 162 faces the bone. The drill sleeve 160 is inserted substantially coaxially along the central axis of the guide sleeve 106a, 106b. The second end 186 of the tip portion 174 of the variable angle tip 112 is then inserted into a variable angle hole of the bone plate such that a central axis of the tip portion 174 is substantially coaxial with a central axis of the variable angle hole. The device 100 is then closed over the bone (i.e., jaws 104a, 104b are moved toward one another such that both the jaws 104a, 104b contact the bone). As the jaws 104a, 104b are closed, the body 162 of the variable angle tip 112 pivots relative to the drill sleeve 160, defining an angle of insertion of a bone fixation element to be inserted through the variable angle hole. In particular, the angle of insertion is determined by the central axis of the drill sleeve 160 relative to the central axis of the tip portion 174. Thus, a guide wire and/or drill tip inserted through the variable angle tip 112, coaxially with the drill sleeve 160 is angled within a predetermined range of angulation relative to the tip portion 174 such that a hole may be drilled into the bone at the desired angle of insertion relative to the central axis of the bone plate.

In a further embodiment, the device 100 further comprises drill guides which may be coupled to the guide sleeves 106a, 106b to facilitate drilling of the bone coaxial to central axes of the guide sleeves 106a, 106b. The drill guides is shaped so that it is insertable through the guide sleeves 106a, 106b (e.g., cylindrical) and includes a lumen extending therethrough such that a drill tip and/or guide wire may be inserted therethrough coaxially with the central axes of the guide sleeves 106a, 106b. In addition, the drill guide may be available in a variety of different sizes (e.g., varying diameters of the lumen) to accommodate drill tips and/or guide wires of various sizes).

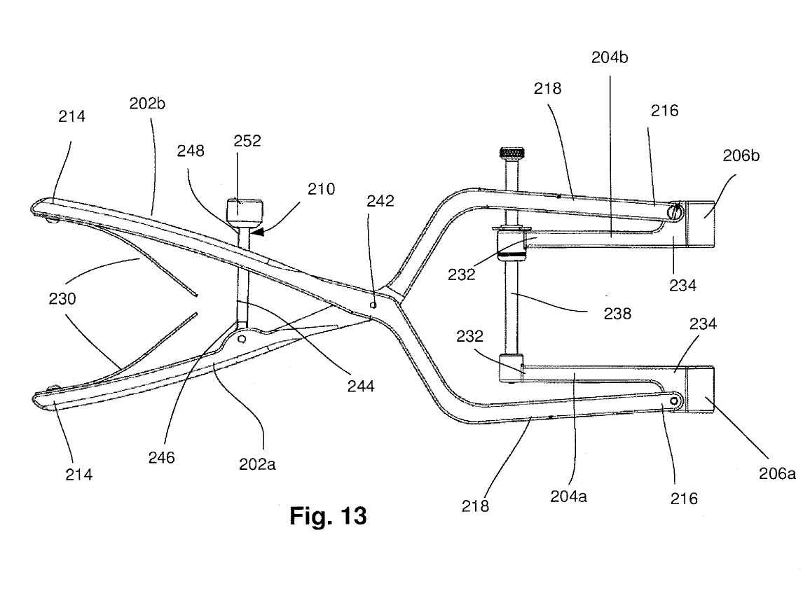

As shown in FIG. 13, a device 200 according to another exemplary embodiment of the present invention is substantially similar to the device 100, comprising a pair of handles 202a, 202b and a pair of jaws 204a, 204b connected to one another such that the jaws 204a, 204b maintain a parallel configuration as the jaws 204a, 204b are moved toward and away from one another to compress fractured portions of bone. Similarly to the device 100, the jaws 204a, 204b include guide sleeves 206a, 206b at distal ends 234 thereof. The handles 202a, 202b cross-over one another and are connected via, for example, a pin 242 such that the handles 202a, 202b pivot relative to one another. Unlike the jaws 104a, 104b, however, the jaws 204a, 204b extend substantially straight from proximal ends 232 to distal ends 234. However, bone gripping portions of the jaws 204a, 204b do not extend distally past distal ends 216 of the handles 202a, 202b. Rather, the jaws 204a, 204b extend between distal portions 218 of the handles 202a, 202b. The distal ends 232 of the jaws 204a, 204b are pivotably attached to distal ends 216 of the handles 202a, 202b while proximal ends 230 of the jaws 204a, 204b are coupled to one another via a sliding post 238. The post 238 of this embodiment is rigidly connected to the first jaw 204a and is slidable through a portion the second jaw 204b. An end of the post 238 is rigidly connected to the proximal end 232 of the first jaw 204a via, for example, a threaded connection such that the post 238 and the first jaw 204a are perpendicular to one another. A length of the post 238 is received through a bushing at the proximal end 232 of the second jaw 204b such that the post 238 is slidable therethrough perpendicular to the second jaw 204b. Thus, as the handles 202a, 202b are moved toward and away from one another, the jaws 204a, 204b maintain a parallel configuration relative to one another through the entire range of motion of the device 200.

The handles 202a, 202b may further including springs 230, biasing the handles apart from one another. Thus, a user may simply release the pressure on the handles 202a, 202b to allow the handles 202a, 202b to move apart from one another. In an alternative embodiment, the handles 202a, 202b are substantially similar to the handles 102a, 102b of the device 100, including finger loops at proximal ends 214 thereof which permit a user to draw the handles away from one another using their fingers. The handles 202a, 202b may also include a locking mechanism 210, substantially similar to the locking mechanism 110 of the device 100. Similarly, the locking mechanism 210 includes a rod 244 extending from a first end 246 pivotable attached to the first handle 202a and slidably attached to the second handle 202b. A nut 252 may be used to lock the handles 202a, 202b relative to one another (e.g., prevent the handles 202a, 202b and jaws 204a, 204b from moving farther apart from one another) such that the jaws 204a, 204b may maintain a compression of fractured bone held therebetween. It will be understood by those of skill in the art that the device 200 may be used in a manner substantially similar to the exemplary technique as described above with respect to the device 100.

It will be apparent to those of skill in the art that various modifications and variations can be made in the structure and the methodology of the present invention, without departing from the spirit or scope of the invention. Thus, it is intended that the present invention cover the modifications and variations of this invention provided that they come within the scope of the appended claims and their equivalents.

What is claimed is:

1. A device for compressing fractured portions of a bone, comprising:
first and second jaws movable relative to one another to compress fractured portions of a bone therebetween, the first and second jaws being connected to one another such that bone gripping portions thereof remain parallel to one another throughout a permitted range of motion of the first and second jaws; and
a first guide sleeve attached to the distal end of the first jaw and a second guide sleeve attached to the distal end of the second jaw, the first and second guide sleeves having central axes coaxial with one another.

2. The device of claim 1, further comprising:
first and second handles connected to the first and second jaws, respectively, such that movement of the first and second handles relative to one another in a first direction moves the first and second jaws toward one another and moving the first and second handles relative to one another in a second direction moves the first and second jaws away from one another.

3. The device of claim 2, further comprising:
a locking mechanism locking a position of the first and second jaws relative to one another.

4. The device of claim 3, wherein the locking mechanism includes a rod extending from a first end attached to the first handle to a second and a nut threadedly engaging the rod, a length of the rod slidably received within a portion of the second handle, the nut movable from an unlocked position adjacent the second end of the rod to a locked position adjacent the second handle.

5. The device of claim 1, wherein at least one of the first and second guide sleeves includes teeth extending along a bone-facing surface thereof to facilitate engagement of bone.

6. The device of claim 1, further comprising:
a variable angle tip couplable to one of the first and second guide sleeves, the variable angle tip including a drill sleeve sized and shaped to be inserted into the first guide sleeve and a body pivotable with respect to the drill sleeve, the body including a tapering channel extending therethrough sized and shaped to engage a variable angle hole in a bone plate and limiting an angulation of the drill sleeve relative to an axis of the variable angle hole to a permitted range of angulation within which a bone fastening element may be lockingly engaged within the variable angle hole.

7. The device of claim 6, wherein the body includes a tip portion sized and shaped to be received within the variable angle hole.

8. The device of claim 7, wherein the body further includes a cap portion releasably coupled to the tip portion, the cap portion pivotably coupling the body to the drill sleeve.

9. The device of claim 1, further comprising:
a drill sleeve couplable to one of the first guide sleeve and the second guide sleeve such that a central axis thereof is coaxial with the central axes of the first and second guide sleeves.

10. The device of claim 1, further comprising:
a spike couplable to one of the first guide sleeve and the second guide sleeve and including a tip configured to engage a bone.

11. A method, comprising:
compressing fractured portions of bone between first and second jaws of a device, the first and second jaws being connected to one another such that bone gripping portions thereof remain parallel to one another throughout a permitted range of motion of the first and second jaws; and
inserting a bone engaging structure into engagement with one of the fractured portions of bone through one of a first guide sleeve attached to a distal end of the first jaw and a second guide sleeve attached to the second jaw, the first and second guide sleeves having central axes coaxial with one another.

12. The method of claim 11, wherein the bone engaging structure is a drill, further comprising the step of drilling a hole in at least one of the fractured portions bone along the central axes of the first and second guide sleeves.

13. The method of claim 11, further comprising:
locking the first and second jaws in a desired position relative to one another by engaging a locking mechanism.

14. The method of claim 13, wherein the locking mechanism includes a rod extending from a first end attached to the first handle to a second and a nut threadedly engaging the rod, a length of the rod slidably received within a portion of the second handle, the nut movable from an unlocked position adjacent the second end of the rod to a locked position adjacent the second handle.

15. The method of claim 11, wherein the bone engaging structure is a k-wire, further comprising the step of inserting the k-wire into at least one of the fractured portions bone along the central axes of the first and second guide sleeves to temporarily fix the portions of bone relative to one another.

16. The method of claim 11, further comprising:
positioning a bone plate over at least one of the fractured portions of bone and advancing the bone engaging structure into engagement with the at least one fractured portion of bone through a hole in the bone plate.

17. The method of claim 16, further comprising:
coupling a variable angle tip to at least one of the first and second guide sleeves by inserting a drill sleeve thereof into one of the first and second guide sleeves, a body of the variable angle tip being pivotable with respect to the drill sleeve, the body including a tapering channel extending therethrough and a tip configured to engage a variable angle hole in the bone plate only in a configuration wherein an angle of the guide sleeve relative to an axis of the variable angle hole is within a permitted range of angulation of the variable angle hole within which a bone fastening element may be locked within the variable angle hole.

18. The method of claim 17, further comprising:
inserting a tip portion of the body into the variable angle hole such that the tip portion is substantially coaxial with the axis of the variable angle hole.

19. The method of claim 18, further comprising:
drawing the first and second jaws together to compress the bone, so that the body pivots with respect to the drill sleeve maintaining the guide sleeves within the predetermined range of angulation as the first and second jaws are drawn together.

20. The method of claim 17, wherein the bone engaging structure is a drill bit, further comprising operating the drill bit to drill a hole into the at least one fractured portion of bone through the variable angle hole.

21. The method of claim 11, further comprising:
coupling a drill sleeve to one of the first guide sleeve and the second guide sleeve such that a central axis thereof is coaxial with the central axes of the first and second guide sleeves and inserting the bone engaging structure into engagement with one of the fractured portions through the drill sleeve.

22. The method of claim 11, further comprising:
coupling a spike to one of the first guide sleeve and the second guide sleeve such that a tip thereof engages one of the fractured portions of bone.
